(19) United States
(12) Patent Application Publication
(10) Pub. No.: US 2003/0060513 A1
(43) Pub. Date: Mar. 27, 2003

(54) Pharmaceutical composition

(76) Inventors: Stephen P. Arneric, Portage, MI (US); Per-Olof Andersson, Whitehouse Station, NJ (US)

Correspondence Address: DINSMORE & SHOHL, LLP, 1900 CHEMED CENTER, 255 EAST FIFTH STREET, CINCINNATI, OH, 45202, US

(21) Appl. No.: 09/965,556

(22) Filed: Sep. 27, 2001

(51) Int. Cl. A61K 31/137

(52) U.S. Cl. 514/648

(57) ABSTRACT

The present invention concerns the field of urology. The invention provides a novel pharmaceutical composition, comprising a pharmaceutically effective combination of (i) a first compound selected from the group consisting of muscarinic receptor antagonists, 5α-reductase inhibitors, and α-adrenergic receptor antagonists, and precursors and pharmaceutically acceptable salts thereof, and (ii) a second compound selected from the group consisting of 5-HT₁ₐ receptor agonists and antagonists, and precursors and pharmaceutically acceptable salts thereof, and optionally a pharmaceutically acceptable carrier or diluent therefor.

There is also provided a method of therapeutical treatment of urinary disorder in a mammal, including man, comprising administering to said mammal, including man, in need of such treatment, a therapeutically effective amount of a composition according to the invention.

### TECHNICAL FIELD

The present invention is within the field of urology. More specifically, it is generally based on the use of a combination of certain agonists and/or antagonists for therapeutical treatment of urinary disorder.

### BACKGROUND OF THE INVENTION

Urinary disorders and symptoms thereof include some or all of the following: urgency, frequency, incontinence, urine leakage, enuresis, dysuria, hesitancy, and difficulty of emptying bladder. In particular, urinary disorders include urinary incontinence, caused by e.g. unstable or overactive urinary bladder.

The term Lower Urinary Tract Symptoms (LUTS) describes a well-recognized medical condition. LUTS include some or all of the following: obstructive urinary symptoms, such as slow urination, dribbling at the end of a urination, inability to urinate and/or the need to strain to urinate at an acceptable rate, or irritative symptoms, such as frequency and/or urgency. These irritative symptoms may result from detrusor overactivity secondary to bladder outlet obstruction resulting from prostatic enlargement or proximal urethral smooth muscle hyperreactivity.

A substantial part (5-10%) of the adult population suffers from urinary incontinence, and the prevalence, particularly of so-called urge incontinence, increases with age. The symptoms of an unstable or overactive bladder comprise urge incontinence, urgency and urinary frequency. Urge incontinence in combination with stress incontinence (mixed incontinence) is frequently encountered by clinicians.

It is assumed that unstable or overactive bladder is caused by uncontrolled contractions of the bundles of smooth muscle fibers forming the muscular coat of the urinary bladder (the detrusor muscle) during the filling phase of the bladder. These contractions are mainly controlled by cholinergic muscarinic receptors, and the pharmacological treatment of unstable or overactive bladder has traditionally been based on muscarinic receptor antagonists.

The reason why the bladder muscle contracts inappropriately is unclear in many cases. For some people it may be due to a problem with the nerve signals that run from the brain to the bladder. Sometimes minor nerve damage is caused by surgery or childbearing. This muscle squeezes or contracts more often than normal and at inappropriate times. Instead of staying at rest as urine fills the bladder, the detrusor contracts while the bladder is filling with urine. This causes a person to feel a sudden and sometimes overwhelming urge to urinate even when the bladder is not full.

Another major urinary disorder is interstitial cystitis. Cystitis is an inflammation of the urinary bladder and associated structures. There is currently no universal effective treatment program. Symptoms from cystitis include urgency for urination, increased frequency of urination and suprapubic pain, usually relieved by voiding, arthritis, spastic colon, low grade fever and irritability. Mammals with cystitis can be significantly disabled and may require surgery. Cystitis can result from e.g. infection, trauma, allergy and malignancy.

U.S. Pat. No. 5,382,600 discloses 2-[(1R)-3-(diisopropylamino)-1-phenylpropyl)-4-methylphenol, also known as N,N-diisopropyl-3-(2-hydroxy-5-methylphenyl)-3-phenylpropylamine, with the generic name of tolterodine, as well as other substituted 3,3-diphenylpropylamines, as being useful to treat urinary incontinence. H Postlind et al, Drug Metabolism and Disposition, 26(4): 289-293 (1998) discloses that tolterodine is a muscarinic receptor antagonist. The active metabolites of tolterodine, as well as other substituted 3,3-diphenylpropylamines, are disclosed in U.S. Pat. No. 5,559,269.

U.S. Pat. No. 4,377,584 discloses the use of finasteride, a 5α-reductase inhibitor, or the treatment of benign prostatic hypertrophy.

U.S. Pat. No. 4,026,894 discloses the use of terazosin, an α-adrenergic receptor antagonist, as an anti-hypertensive agent. α-adrenergic receptor antagonists relax smooth muscle.

U.S. Pat. No. 5,990,114 discloses the use of certain 5-HT₁ₐ receptor antagonists for the treatment of urinary incontinence.

Despite the above advances in the art, it is desirable to develop novel pharmaceutical compositions that further improve the quality of life for a large number of individuals.

### SUMMARY OF THE INVENTION

For these and other purposes, it is an object of the present invention to provide a novel pharmaceutical composition for treating urinary disorder in a mammal, including man, which composition inhibits, or suppresses, unstable bladder contractions and diminishes problems associated with incomplete bladder emptying.

It is also an object of the present invention to provide a novel method of treating urinary disorder in a mammal, including man, which method effectively inhibits, or suppresses, unstable bladder contractions and diminishes problems associated with incomplete bladder emptying.

For these and other objects that will be evident from the following disclosure, the present invention provides a novel pharmaceutical composition, comprising a pharmaceutically effective combination of

(i) a first compound selected from the group consisting of muscarinic receptor antagonists, 5α-reductase inhibitors, and α-adrenergic receptor antagonists, and precursors and pharmaceutically acceptable salts thereof, and

(ii) a second compound selected from the group consisting of 5-HT₁ₐ receptor agonists and antagonists, and precursors and pharmaceutically acceptable salts thereof,

and optionally a pharmaceutically acceptable carrier or diluent therefor.

The invention is based on the insight that a combination of at least one compound selected from the group consisting of muscarinic receptor antagonists, 5α-reductase inhibitors, and α-adrenergic receptor antagonists, with a 5-HT₁ₐ-agonist or -antagonist produces a favorable simultaneous effect on bladder contractility and bladder storage, as will be described more below.

In a preferred embodiment of the composition according to the invention, said first compound is a muscarinic receptor antagonist, or a precursor or a pharmaceutically acceptable salt thereof.

In a more preferred embodiment of the composition according to the invention, said muscarinic receptor antagonist is a substituted 3,3-diphenylpropylamine. Among substituted 3,3-diphenylpropylamines with muscarinic receptor antagonist activity are those referred to in the background of the invention.

In an even more preferred embodiment of the composition according to the invention, said substituted 3,3-diphenylpropylamine is selected from the group consisting of tolterodine and hydroxytolterodine. Preferably, said substituted 3,3-diphenylpropylamine is tolterodine. In the most preferred embodiment of the composition according to the invention, said first compound is tolterodine L-tartrate.

In another preferred embodiment of the composition according to the invention, said muscarinic receptor antagonist is selected from oxybutynin and active derivatives thereof. Among active derivatives thereof is its active metabolite N-desethyloxybutynin. Preferably, said muscarinic receptor antagonist is oxybutynin.

In yet another preferred embodiment of the composition according to the invention, said muscarinic receptor antagonist is selected from darifenacin and active derivatives thereof. Among active derivatives thereof is its active 3′-hydroxyl metabolite. Preferably, said muscarinic receptor antagonist is darifenacin.

In one preferred embodiment of the composition according to the invention, said first compound is present in an amount of from about 0.1 mg to about 100 mg.

In a preferred embodiment of the composition according to the invention, said second compound is a neutral 5-HT₁ₐ receptor antagonist.

In one preferred embodiment of the composition according to the invention, said second compound is present in an amount of from about 0.1 mg to about 1 g.

In another preferred embodiment of the composition according to the invention, said first compound and said second compound are maintained in the same delivery vehicle.

In yet another preferred embodiment of the composition according to the invention, said first compound and said second compound are maintained in different delivery vehicles.

In a preferred embodiment of the composition according to the invention, said composition is for treating urinary disorder in a mammal, including man. In a more preferred embodiment of the composition according to the invention, said disorder is selected from the group consisting of lower urinary tract symptoms, unstable or overactive urinary bladder, bladder outflow obstruction, urinary incontinence, particularly stress incontinence, and interstitial cystitis.

In another preferred embodiment of the composition according to the invention, said composition is for treating depression in said mammal, which depression is concomitant with said urinary disorder.

Furthermore, the present invention provides use of the composition according to the invention for the manufacture of a medicament for therapeutical treatment of urinary disorder in a mammal, including man. In a preferred embodiment of the use according to the invention, the medicament is for treatment of depression in said mammal, which depression is concomitant with said urinary disorder.

Furthermore, the present invention provides a method of therapeutical treatment of urinary disorder in a mammal, including man, comprising administering to said mammal, including man, in need of such treatment, a therapeutically effective amount of a composition according to the invention.

In a preferred embodiment of the method according to the invention, said disorder is selected from the group consisting of lower urinary tract symptoms, unstable or overactive urinary bladder, bladder outflow obstruction, urinary incontinence, particularly stress incontinence, and interstitial cystitis.

In another preferred embodiment of the method according to the invention, said method is also for treating depression in said mammal, which depression is concomitant with said urinary disorder.

In a preferred embodiment of the method according to the invention, said composition is administered rectally, intravaginally, topically, orally, sublingually, intranasally, transdermally or parenterally.

In another preferred embodiment of the method according to the invention, said first compound and said second compound of said composition are simultaneously administered.

In yet another preferred embodiment of the method according to the invention said first compound and said second compound of said composition are concomitantly administered.

Finally, the present invention provides a pharmaceutical kit for therapeutical treatment of urinary disorder in a mammal, including man, comprising

(i) a first container comprising a first compound as described above

(ii) a second container comprising a second compound as described above, and

(iii) instructions for use of the kit.

### DESCRIPTION OF THE INVENTION

In describing the prefer-red embodiment, certain terminology will be utilized for the sake of clarity. Such terminology is intended to encompass the recited embodiments, as well as all technical equivalents that operate in a similar manner for a similar purpose to achieve a similar result. To the extent that any pharmaceutically active compound is disclosed or claimed, it is expressly intended to include all active metabolites produced in vivo, and, is expressly intended to include all enantiomers, isomers or tautomers where the compound is capable of being present in its enantiomeric, isomeric or tautomeric form.

The present invention provides a novel composition, which is a combination of

at least one muscarinic receptor antagonist or 5α-reductase inhibitor or α-adrenergic receptor antagonist or norepinephrine and/or serotonin reuptake inhibitor and a 5-HT₁ₐ agonist or antagonist. The inventive composition is useful for the treatment of urinary disorder.

A particularly preferred composition for the treatment of urinary disorder is a combination of an anti-muscarinic agent and a neutral 5-HT₁ₐ-antagonist.

According to the invention, it has now surprisingly and inventively been found that treatment with a combination of an anti-muscarinic agent and a neutral 5-HT₁ₐ-antagonist produces a simultaneous effect on bladder contractility and bladder storage.

Anti-muscarinic treatment acts on the effector organ by inhibiting the response to efferent impulses from the central nervous system. Thus, anti-muscarinic treatment inhibits unstable bladder contractions during the filling phase but also inhibits the contractions elicited during the elimination phase, especially at higher doses, thereby resulting in a decrease in micturition pressure, eventually leading to the negative consequence of incomplete bladder emptying. This effect limits the possibilities of otherwise acceptable dosing of these agents. Furthermore, anti-muscarinic treatment leads to side-effects outside of the urogenital systems, mainly due to blockade of muscarinic receptors in other tissues such as the salivary glands, the gut, and the CNS, leading to side effects such as dry mouth, constipation, and confusion, respectively. To some extent, these side effects have been reduced by the introduction of newer anti-muscarinic agents such as tolterodine with selectivity for bladder smooth muscle. However, even bladder-selective anti-muscarinic agents will always be limited as a treatment of overactive bladder by their effect on the micturition contraction described above.

The effects of anti-muscarinic agents have been studied in a range of animal models and they have consistently been shown to reduce the amplitude of voiding or micturition contraction without direct effects on bladder capacity. For these agents, the effects on bladder capacity have always been shown to be secondary to a significant decrease in micturition pressure.

No clinically available agents have any direct effect on the storage function of the bladder. However, it has now been realized that a combination of 5-HT₁ₐ-agonists or-antagonists, particularly neutral 5-HT₁ₐ-antagonists, and antimuscarinic agents or 5α-reductase inhibitors or α-adrenergic receptor antagonists or norepinephrine and/or serotonin reuptake inhibitors, particularly antimuscarinic agents, increases bladder capacity without negative consequences on bladder contractility.

Importantly, in models for the evaluation of the effects of an anti-muscarinic agent on bladder contractility, simultaneous administration of a neutral 5-HT-antagonist with an anti-muscarinic does not attenuate the effects of the anti-muscarinic agent on bladder contractility.

Furthermore, in models used for evaluation of the effects of neutral 5-HT₁ₐ antagonists on bladder capacity and inhibition of the micturition reflex, simultaneous administration of an anti-muscarinic agent with a neutral 5-HT₁ₐ-antagonist does not attenuate the effects of the 5-HT₁ₐ-antagonist on bladder capacity or its effect on the micturition reflex.

The muscarinic receptor antagonists, or antimuscarinic agents, useful in the pharmaceutical compositions of this invention include, but are not limited to, non-selective agents, bladder-selective agents and muscarinic M3 receptor-selective agents. Examples of muscarinic receptor antagonists include, but are not limited to, tolterodine and active metabolites thereof, such as hydroxytolterodine, YM905, propiverine, oxybutynin, trospium, propantheline, darifenacin, temiverine, and ipratropium, as well as pharmaceutically acceptable salts thereof. YM905 is butanedioic acid, compd. with (1S)-(3R)-1-azabicyclo[2.2.2]oct-3-yl 3,4-dihydro-1-phenyl-2(1H)-isoquinolinecarboxylate (1:1) (9CI). Propiverine is 1-methyl-4-piperidyl .alpha.,.alpha.-diphenyl-.alpha.-(n-propoxy)acetate and is disclosed in East German Patent 106,643 and in CAS 82-155841s (1975). Oxybutynin is 4-(diethylamino)-2-butynylalphaphenylcyclohexaneglycolate and is disclosed in UK Patent 940,540. Trospium is 3alpha-hydroxyspiro[1alphaH,5alphaH-nortropane-8,1′pyrrolidinium]chloride benzilate and is disclosed in U.S. Pat. No. 3,480,623. Darifenacin is (S)-2-{1-[2-(2,3-dihydrobenzofuran-5-yl)ethyl]-3-pyrrolidinyl}-2,2-diphenyl-acetamide, and is disclosed in U.S. Pat. No. 5,096,890. Temiverine is benzeneacetic acid, .alpha.-cyclohexyl-.alpha.-hydroxy-, 4-(diethylamino)-1,1-dimethyl-2-butynyl ester and is disclosed in U.S. Pat. No. 5,036,098. Ipratropium is 8-isopropylnoratropine methobromide and is disclosed in U.S. Pat. No. 3,505,337.

Preferred muscarinic receptor antagonists may be selected from substituted 3,3-diphenylpropylamines (such as those disclosed in U.S. Pat. No. 5,382,600) with antimuscarinic activity, as well as pharmaceutically acceptable salts thereof. Preferred muscarinic receptor antagonists include, but are not limited to tolterodine and hydroxytolterodine, oxybutynin and active derivatives thereof, such as N-desethyloxybutynin, and darifenacin and active derivatives thereof, such as its 3′-hydroxyl metabolite, as well as pharmaceutically acceptable salts thereof.

The 5α-reductase inhibitors useful in the pharmaceutical compositions of this invention include, but are not limited to, finasteride (U.S. Pat. No. 4,377,584), dutasteride (U.S. Pat. No. 5,565,467), epristeride (U.S. Pat. No. 5,017,568), and turosteride (U.S. Pat. No. 5,155,107), as well as pharmaceutically acceptable salts thereof.

The α-adrenergic receptor antagonists useful in the pharmaceutical compositions of this invention include, but are not limited to, terazosin (U.S. Pat. No. 4,026,894), doxazosin (U.S. Pat. No. 4,188,390), prazosin (U.S. Pat. No. 3,511,836), bunazosin (U.S. Pat. No. 3,920,636), indoramin (U.S. Pat. No. 3,527,761), alfuzosin (U.S. Pat. No. 4,315,007), abanoquil (U.S. Pat. No. 4,686,228), naftopidil (U.S. Pat. No. 3,997,666), phentolamine, tamsulosin (U.S. Pat. No. 4,703,063), trazodone, dapiprazole, phenoxybenzamine, idazoxan (U.S. Pat. No. 4,818,764), efaroxan (U.S. Pat. No. 4,411,908), yohimbine, dibenzamine, trimazosin, tolazoline, corynthanine, rauwolscine, tamsulosin, and piperoxan, as well as pharmaceutically acceptable salts thereof.

The norepinephrine and/or serotonin reuptake inhibitors useful in the pharmaceutical compositions of this invention include, but are not limited to, duloxetine (U.S. Pat. No. 4,956,388) and reboxetine.

The selection of the dosage of the first compound is that which can provide relief to the patient. As is well known, the dosage and administrative regimen (i.e., one, two, three or more administrations per day) of this compound depends on several factors such as the potency of the selected specific compound, the mode of administration, the age and weight of the patient, the severity of the condition to be treated, and the like. This is considered to be within the skill of the artisan, and one can review the existing literature on the components to determine optimal dosing.

When the first compound is an antimuscarinic agent, it is preferred that the average adult daily dosage of the first compound is from about 0.05 mg to about 5 mg per kilogram of body weight, administered in one or more doses, e.g. containing from about 0.05 mg to about 250 mg each.

When the first compound is a 5α-reductase inhibitor, it is preferred that the first compound is present in an amount ranging from about 2 mg to about 20 mg, preferably about 5 mg per dose.

When the first compound is an α-adrenergic receptor antagonist, it is preferred that the first compound is present in an amount ranging from about 1 mg to about 25 mg, and preferably about 10 mg per dose.

The 5-HT₁ₐ receptor agonists and antagonists useful in the pharmaceutical compositions of this invention include, but are not limited to, compounds that act on the central nervous system by binding to 5-HT receptors of the 5-HT₁ₐ subtype. Non-limiting examples of 5-HT₁ₐ receptor antagonists are WAY-100,635, i.e. cyclohexanecarboxamide, N-[2-[4-(2-methoxyphenyl)-1-piperazinyl]ethyl]-N-2-pyridinyl-, trihydrochloride, robalzotan, i.e. (3R)-3-(dicyclobutylamino)-8-fluoro-3,4-dihydro-2H-1-benzopyran-5-carboxamide, and LY426965, i.e. [(2S)-(+)-1-cyclohexyl-4-[4-(2-methoxyphenyl)-1-piperazinyl]2-methyl-2-phenyl-1-butanone monohydrochloride]. In general, the compounds selectively bind to receptors of the 5-HT₁ₐ subtype to a much greater extent than they bind to other receptors, such as α₁ and D₂ receptors. Moreover, they exhibit activity as 5-HT₁ₐ-agonists or -antagonists in pharmacological testing. The 5-HT₁ₐ receptor agonists and antagonists of the invention can be used for the treatment of CNS disorders, such as anxiety in mammals, particularly humans. They may also be used as antidepressants, hypotensives, as agents for regulating the sleep/wake cycle, feeding behavior and/or sexual function, for treating cognition disorders, and for treating neuromuscular dysfunction of the lower urinary tract, particularly those involving micturition (urination), such as dysuria, incontinence, and enuresis.

A neutral antagonist is a compound that binds to a receptor, is devoid of intrinsic activity at the receptor, but blocks the receptor-mediated functional activity elicited by an agonist. In this respect, an agonist is defined as a compound that binds to a receptor and activates a receptor-mediated functional response such as, but not limited to, 5-HT₁ₐ-mediated inhibition of adenylyl cyclase activity or activation of potassium channels.

The dosage and administrative regimen (i.e., one, two, three or more administrations per day) of the second compound depends on the factors referred to in connection with the dosage selection of the first compound. The average adult daily dosage of the second compound is from about 1 μg to about 10 mg per kilogram of body weight, administered in one or more doses, e.g. containing from about 50 μg to about 1 g each. Pediatric dosages may be less.

Examples of pharmaceutically acceptable salts for use in the composition according to the invention include, but are not limited to, acetate, benzoate, hydroxybutyrate, bisulfate, bisulfite, bromide, butyne-1,4-dioate, carpoate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogenphosphate, naphthalene-1-sulfonate, naphthalene-2-sulfonate, oxalate, phenylbutyrate, phenylproionate, phosphate, phthalate, phylacetate, propanesulfonate, propiolate, propionate, pyrophosphate, pyrosulfate, sebacate, suberate, succinate, sulfate, sulfite, sulfonate, tartrate, xylenesulfonate, and the like.

Compositions of the present invention can conveniently be administered in a pharmaceutical composition containing the active compounds in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). To the extent necessary for completion, this reference is hereby incorporated by reference. The compositions of the present invention can be administered parenterally (for example, by intravenous, intraperitoneal, subcutaneous or intramuscular injection), topically, orally, sublingually, transdermally, intranasally, intravaginally, or rectally, with oral administration being particularly preferred.

For oral therapeutic administration, the inventive composition may be combined with one or more excipients and used in the form of ingestible tablets, buccal tablets, troches, capsules, elixirs, suspensions, syrups, wafers, chewing gums, foods and the like. Such compositions and preparations preferably contain at least 0.1% of active compounds. The percentage of the compositions and preparations may, of course, be varied and may conveniently be between about 0.1 to about 100% of the weight of a given unit dosage form. The amount of active compounds in such therapeutically useful compositions is such that effective dosage levels will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative, and one skilled in the art could envision other binders, excipients, sweetening agents and the like. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceuticaly acceptable and substantially non-toxic in the amounts employed. In addition, the active components may be incorporated into sustained-release preparations and devices including, but not limited to, those relying on osmotic pressures to obtain a desired release profile. Once daily formulations for each of the active components are specifically included.

The inventive composition, containing the two, or more, active compounds, may be administered in the same physical form or concomitantly according to the above-described dosages and in the above-described delivery vehicles. The dosages for each active compound can be measured separately and can be given as a single combined dose or given separately. They may be given at the same or at different times as long as both actives are in the patient at one time over a 24-hour period. Concomitant or concurrent administration means that the patient takes one drug within about 5 minutes of taking the other drug.

The present invention also provides a pharmaceutical kit for therapeutical treatment of urinary disorder in a mammal, including man. In analogy with the composition, the kit comprises a first container comprising a first compound as described above, a second container comprising a second compound as described above, and instructions for use of the kit.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

The inventive composition is to be used in the treatment of urinary disorders. In particular, the composition is useful for treating LUTS or incontinence of any type, e.g. stress incontinence, genuine stress incontinence, and mixed incontinence. Stress urinary incontinence is a symptom describing involuntary loss of urine on carrying out any activity that raises intra-abdominal pressure such as coughing or sneezing. Stress incontinence is also a clinical sign, that is the observation by a care giver of a jet of urine escaping from the urethral meatus (opening) when the patient coughs or strains. Genuine Stress Incontinence (urge incontinence) is the pathological diagnosis of an incompetent urethral sphincter as diagnosed by Urodynamic testing. Mixed incontinence is stress incontinence in combination with urge incontinence. The latter is a part of the symptom complex of the Overactive Bladder. Retention may be due to outflow obstruction (e.g., high urethral pressure), poor detrusor (bladder muscle) contractility or lack of coordination between detrusor contraction and urethral relaxation. The inventive drug combination can be used in connection with stress incontinence, urge incontinence or mixed incontinence.

The composition according to the invention is also to be used in the treatment of interstitial cystitis.

In a situation where anti-muscarinic treatment of a urinary disorder is limited by an increase in residual urine, treatment can be augmented by the addition of a neutral 5-HT₁ₐ antagonist. This situation is especially likely to occur in patients with overactive bladder secondary to bladder outflow obstruction, e.g. due to prostate enlargement.

In other cases, anti-muscarinic treatment might be limited by intolerable side effects, such as dry mouth. In such a case, the anti-muscarinic dose might be reduced but efficacy maintained by the addition of a neutral 5-HT₁ₐ antagonist. This combination allows the use of anti-muscarinic agents that are not selective for the bladder in a situation where these agents are preferred over other, more bladder selective, agents.

In another situation, treatment with a neutral 5-HT₁ₐ antagonist might be limited due to absence of an effect on bladder contractility. In such a case, addition of an anti-muscarinic agent brings additional efficacy. Such a situation might be patients with bladder hyperreflexia, a condition known to be associated with increased reflex bladder contractions.

In yet another situation, the effectiveness of a neutral 5-HT₁ₐ antagonist might be limited by side effects. In such a case, adjustment of the dose of the 5-HT antagonist, and thereby its effectiveness can be compensated for by the addition of an anti-muscarinic agent.

The novel composition is considered to provide rapid relief to those suffering from the above diseases or disorders with a minimal amount of deleterious side effects.

The invention is described in greater detail by the following non-limiting examples.

### EXAMPLES

### Example 1

A pharmaceutical composition is prepared by combining tolterodine with a neutral 5-HT₁ₐ receptor antagonist in a pharmaceutically acceptable carrier. The composition contains between about 0.05 mg to about 4 mg of tolterodine per kilogram of patient body weight (for example, 3 mg to 240 mg tolterodine for a person weighing 60 kg) and between about 0.01 mg to about 1 mg of neutral 5-HT₁ₐ receptor antagonist per kilogram of patient body weight. The composition is administered to a patient for the treatment of incontinence, and particularly stress incontinence, urge incontinence or mixed incontinence.

### Example 2

A first pharmaceutical composition is prepared by combining a neutral 5-HT₁ₐ receptor antagonist in a pharmaceutically acceptable carrier such that it can deliver between about 0.5 mg to about 50 mg on a daily basis. A second pharmaceutical composition is prepared by combining tolterodine in a pharmaceutically acceptable carrier such that it can deliver between about 0.05 mg to about 4 mg of tolterodine per kilogram of patient body weight on a daily basis.

The first composition is administered to a patient suffering from one or more forms of incontinence once, twice, three times, four times or six times daily such that the daily dosage is between about 0.5 mg to about 50 mg. The second composition is administered to the same patient at the same time as the administration of the first composition or any time within 24 hours of the administration of the first composition once, twice, three times, four times or six times daily such that the daily dosage is between about 0.05 mg to about 4 mg of tolterodine per kilogram of patient body weight. Alternatively, the second composition could first be administered, followed by the administration of the first composition as disclosed at the same time, or within 24 hours thereof.

### Example 3

A pharmaceutical composition is prepared by combining a 5α-reductase inhibitor with a neutral 5-HT₁ₐ receptor antagonist in a pharmaceutically acceptable carrier. The composition contains between about 2 mg to about 20 mg of 5α-reductase inhibitor and between about 0.5 mg to about 50 mg of neutral 5-HT₁ₐ receptor antagonist. The composition is administered to a patient for the treatment of urinary disorder.

### Example 4

A pharmaceutical composition is prepared by combining an α-adrenergic receptor antagonist with a neutral 5-HT₁ₐ receptor antagonist in a pharmaceutically acceptable carrier. The composition contains between about 1 mg to about 25 mg of α-adrenergic receptor antagonist and between about 0.5 mg to about 50 mg of neutral 5-HT₁ₐ receptor antagonist. The composition is administered to a patient for the treatment of urinary disorder.

Having described the invention in detail and by reference to the preferred embodiments thereof, it will be apparent that modifications and variations are possible without departing from the scope of the appended claims.

1. A pharmaceutical composition comprising a pharmaceutically effective combination of
(i) a first compound selected from the group consisting of muscarinic receptor antagonists, 5α-reductase inhibitors, and α-adrenergic receptor antagonists, and precursors and pharmaceutically acceptable salts thereof, and
(ii) a second compound selected from the group consisting of 5-HT₁ₐ receptor agonists and antagonists, and precursors and pharmaceutically acceptable salts thereof,
and optionally a pharmaceutically acceptable carrier or diluent therefor.

2. A pharmaceutical composition according to claim 1, wherein said first compound is a muscarinic receptor antagonist, or a precursor or a pharmaceutically acceptable salt thereof.

3. A composition according to claim 2, wherein said muscarinic receptor antagonist is a substituted 3,3-diphenylpropylamine.

4. A composition according to claim 3, wherein said substituted 3,3-diphenylpropylamine is selected from the group consisting of tolterodine and hydroxytolterodine.

5. A composition according to claim 4, wherein said substituted 3,3-diphenylpropylamine is tolterodine.

6. A composition according to claim 5, wherein said first compound is tolterodine L-tartrate.

7. A composition according to claim 2, wherein said muscarinic receptor antagonist is selected from oxybutynin and active derivatives thereof, such as N-desethyloxybutynin.

8. A composition according to claim 7, wherein said muscarinic receptor antagonist is oxybutynin.

9. A composition according to claim 2, wherein said muscarinic receptor antagonist is selected from darifenacin and active derivatives thereof, such as its 3′-hydroxyl metabolite.

10. A composition according to claim 9, wherein said muscarinic receptor antagonist is darifenacin.

11. A composition according to any one of claims 1-10, wherein said first compound is present in an amount of from about 0.1 mg to about 100 mg.

12. A composition according to any one of claims 1-11, wherein said second compound is a neutral 5-HT₁ₐ receptor antagonist.

13. A composition according to any one of claims 1-12, wherein said second compound is present in an amount of from about 0.1 mg to about 1 g.

14. A composition according to any one of claims 1-13, wherein said first compound and said second compound are maintained in the same delivery vehicle.

15. A composition according to any one of claims 1-13, wherein said first compound and said second compound are maintained in different delivery vehicles.

16. A composition according to any one of claims 1-15, which is for treating urinary disorder in a mammal, including man.

17. A composition according to claim 16, wherein said disorder is lower urinary tract symptoms.

18. A composition according to claim 16, wherein said disorder is unstable or overactive urinary bladder.

19. A composition according to claim 16, wherein said disorder is bladder outflow obstruction.

20. A composition according to claim 16, wherein said disorder is urinary incontinence.

21. A composition according to claim 20, wherein said disorder is stress incontinence.

22. A composition according to claim 16, wherein said disorder is interstitial cystitis.

23. A composition according to any one of claims 16-22, which is for treating depression in said mammal, which depression is concomitant with said urinary disorder.

24. Use of a pharmaceutical composition according to any one of claims 1-15 for the manufacture of a medicament for therapeutical treatment of urinary disorder in a mammal, including man.

25. Use of a pharmaceutical composition according to claim 24, wherein said disorder is lower urinary tract symptoms.

26. Use of a pharmaceutical composition according to claim 24, wherein said disorder is unstable or overactive urinary bladder.

27. Use of a pharmaceutical composition according to claim 24, wherein said disorder is bladder outflow obstruction.

28. Use of a pharmaceutical composition according to claim 24, wherein said disorder is urinary incontinence.

29. Use of a pharmaceutical composition according to claim 28, wherein said disorder is stress incontinence.

30. Use of a pharmaceutical composition according to claim 24, wherein said disorder is interstitial cystitis.

31. Use of a pharmaceutical composition according to any one of claims 24-30, wherein the medicament is for treatment of depression in said mammal, which depression is concomitant with said urinary disorder.

32. A method of therapeutical treatment of urinary disorder in a mammal, including man, comprising
administering to said mammal, including man, in need of such treatment, a therapeutically effective amount of a composition according to any one of claims 1-15.

33. A method of therapeutical treatment according to claim 32, wherein said disorder is lower urinary tract symptoms.

34. A method of therapeutical treatment according to claim 32, wherein said disorder is unstable or overactive urinary bladder.

35. A method of therapeutical treatment according to claim 32, wherein said disorder is bladder outflow obstruction.

36. A method of therapeutical treatment according to claim 32, wherein said disorder is urinary in continence.

37. A method of therapeutical treatment according to claim 36, wherein said disorder is stress incontinence.

38. A method of therapeutical treatment according to claim 32, wherein said disorder is interstitial cystitis.

39. A method of therapeutical treatment according to any one of claims 32-38, which is also for treatment of depression in said mammal, which depression is concomitant with said urinary disorder.

40. A method of therapeutical treatment according to any one of claims 32-39, wherein said composition is administered rectally, intravaginally, topically, orally, sublingually, intranasally, transdermally or parenterally.

41. A method of therapeutical treatment according to any one of claims 32-40, wherein said first compound and said second compound of said composition are simultaneously administered.

42. A method of therapeutical treatment according to any one of claims 32-40, wherein said first compound and said second compound of said composition are concomitantly administered.

43. A pharmaceutical kit for therapeutical treatment of urinary disorder in a mammal, including man, comprising
(i) a first container comprising a first compound according to any one of claims 1-10,
(ii) a second container comprising a second compound according to claim 1 or 12, and optionally
(iii) instructions for use of the kit.
